(19) United States
(12) Patent Application Publication
(10) Pub. No.: US 2005/0287621 A1
(43) Pub. Date: Dec. 29, 2005

(54) Chip and apparatus for acetylcholinesterase inhibitor detection and method for using thereof

(75) Inventor: Jing-Hsiang Hsu, Keelung City (TW)

Correspondence Address: BACON & THOMAS, PLLC, 625 SLATERS LANE, FOURTH FLOOR, ALEXANDRIA, VA, 22314, US

(73) Assignee: HSU, Jing-Hsiang, Keelung City (TW)

(21) Appl. No.: 10/941,840

(22) Filed: Sep. 16, 2004

(30) Foreign Application Priority Data
Jun. 29, 2004 (TW) 93119013

(51) Int. Cl. C12Q 1/46; C12M 1/34

(52) U.S. Cl. 435/20; 435/287.1

(57) ABSTRACT

A chip and an apparatus for acetylcholinesterase inhibitor detection and a method for using thereof are provided. The chip of the invention comprises a substrate and a film, coated on said substrate, including a molecule with a specific ability to combine with an acetylcholinesterase inhibitor. The present invention utilizes the surface modification technology and cooperates with optical precision instruments to provide an acetylcholineases inhibitor detection chip and an apparatus with high sensitivity and suitable for both liquid and gas detection.

### BACKGROUND OF THE INVENTION

1. Field of the Invention

The present invention relates to a chip, an apparatus for acetylcholinesterase inhibitor detection and a method for using thereof.

2. Description of the Related Art

Acetylcholine (Ach) is a general neurotransmitter in central and peripheral nervous systems and released at the terminals of nerve fibres innervating skeletal muscle, at synapses in autonomic ganglia and by the post-ganglionic parasympathetics. It is the primary chemical responsible for the transmission of nerve impulses across the synapse of two neurons. After the impulse is transmitted across the synapse, the acetylcholine is broken down by acetylcholinesterase, an enzyme for decomposing acetylcholine. When this occurs, the synapse is “cleared” and ready to receive a new transmission. Once the activity of acetylcholinesterase became abnormal, the accumulations of acetylcholine were affected, then intervening nervous systems. Therefore, in pharmacology, nerve agents all belong to acetylcholinesterase inhibitors.

Nerve agents refer to chemical agents that intervene nervous systems, causing physiological abnormal and inducing toxic effect in organisms. It usually features rapid development of effects and acute toxicity. Most nerve agents belong to organophosphorus chemicals. Organophosphorus chemicals are originally used as pesticides to control pests and protect croppers. German chemists first synthesized the nerve agents, called Tabun (GA), in 1936. Other nerve agents, such as Sarin (GB), Soman (GD) and VX, were synthesized subsequently. All of them are organoposphorus chemicals.

The mechanism of nerve agents is that the nerve agents compete “binding sites” of acetylcholinesterases with acetylcholine. Because the combination of nerve agents and acetylcholinesterases is easier than that of acetylcholine and acetylcholinesterases due to the chemical structure of the nerve agent, the acetylcholinesterase was phosphorylated, losing the function of acetylcholine hydrolysis, resulting in the accumulation of acetylcholine released from cholinegic neuron, finally causing abnormal symptoms in physiology, such as spasm, dyspnea, spewing, abdominal pain, coma or paralysis, in other words, causing intoxication.

The nerve agent detection apparatus used in recent years include M256 series detector kit, ABC-M8 detector paper, ChemPro 100 and M90 detector. However, M256 series detector kit and ABC-M8 detector paper detect nerve agents based on color presentation that is too simple to provide high sensitivity and credibility. For example, the M256 series detector kit changes its color when contacting with smoke bombs, providing low accuracy; and ABC-M8 detector papers are used only for liquid agents detection, not for gas agents. The detectors of ChemPro 100 and M90 detect agents based on open loop ion mobility spectrometry technology: the detected materials are first compared with the built-in chemical database and users are then informed of chemical category through signal transducer. However, ChemPro 100 and M90 detect gas agents only and the isotope Americium 241 used in ChemPro 100 and M90 may affect the health of users.

In addition to the nervous toxicity, the acetylcholinesterase inhibitors can be used to treat some acetylcholinesterase-related disorders. For example, acetylcholinesterase inhibitors, such as Aricept, Exelon and Reminyl, can be used to treat Alzheimer's disease. It is effective for a patient to slow the disease development by taking those medicines from primary stage to middle stage.

Nevertheless, to be nerve agents or medicines, the presence of acetylcholinesterase inhibitors influences nervous systems in human. Thus, there is a need for developing an accurate apparatus and method for detecting acetycholinesterase inhibitors.

### SUMMARY OF THE INVENTION

To address the drawbacks of traditional nerve agent detectors and the needs of screening medicines with acetycholinesterase inhibition, the present invention utilizes the surface modification technology and cooperates with optical precision instruments to provide a chip for acetylcholineases inhibitor detection with high sensitivity and suitable for both liquid and gas detection.

In one aspect, the present invention provides a chip for acetylcholinease inhibitor detection, comprising:
a substrate; and
a film including a molecule with a specific ability to combine with an acetylcholinesterase inhibitor and said film is coated on said substrate.

In another aspect, the present invention provides an apparatus for acetylcholinesterase inhibitor detection, comprising:
an acetylcholinesterase inhibitor detection chip including a substrate and a film coated on said substrate, wherein said film including a molecule with a specific ability to combine with an acetylcholinesterase inhibitor; and
a detection element for detecting the surface variation of said acetylcholinesterase inhibitor detection chip.

In a further aspect, the present invention provides a method for detecting acetylcholinesterase inhibitors, comprising the following steps of:
(a) providing an acetylcholinesterase inhibitor detection chip including a substrate and a film coated on said substrate, wherein said film including a molecule with a specific ability to combine with a acetylcholinesterase;
(b) contacting said acetylcholinesterase inhibitor detection chip with a sample;
(c) detecting the surface variation of said acetylcholinesterase inhibitor detection chip; and
(d) determining whether an acetylcholinesterase inhibitor exists in the sample.

The present invention utilizes the surface modification technology to provide an acetylcholinesterase inhibitor detection chip, and cooperates with surface plasmon resonance (SPR) or quartz crystal microbalance (QCM) to determine the presence of acetylcholinesterase inhibitors and quantify concentrations. Because trace substances can be measured by SPR or QCM from 10⁻⁶ g to 10⁻¹² g, real-time qualitative and quantitative detection for acethycholinesterase inhibitor could be accomplished by the combination of an acetylcholinesterase inhibitor detection chip of the invention with a SPR or a QCM, and such combination can detect both liquid and gas samples. The present invention provides an acetylcholinesterase inhibitor detection chip and apparatus with good efficacy; moreover, the chip and apparatus of the invention can be used to screen aceylcholinesterase-releated medicines.

### BRIEF DESCRIPTION OF DRAWINGS

FIG. 1 is a scheme diagram of the method for detecting acetylcholinesterase inhibitors of the invention.

FIG. 2 is a scheme diagram of the method for modifying the gold surface with dextran.

### DETAILED DESCRIPTION OF THE INVENTION

In the present invention, the term “acetylcholinesterase inhibitor” or “acetylchooinesterase inhibition molecule” refers to any natural or synthesized compounds or molecules that influence the acetylcholine hydrolysis function of acetylcholinesterase. The examples of the acetylcholinesterase inhibitor are, but not limited to, nerve agents, organophosphorus insecticides or medicines with acetylcholinsterase inhibition. Generally, the acetylcholinesterase inhibitor is considered as a nerve agent. It is because that an acetylcholinesterase inhibitor can be combined with an acetylcholinesterase that causes the loss of acetylcholine hydrolysis for acetylcholinesterase, and the normal mechanisms of nervous systems are interrupted. However, proper amounts of acetylcholinesterase inhibitors can be used as medicines for treating some acetylcholinesterase-related disorders.

In the present invention, the term “nerve agent” refers to any chemical agent with a property of nervous system intervention that causes physiological abnormal and induces toxic effect in organisms. The examples of nerve agents are, but limited to, synthesized agents such as organophosphorus agent, Sarin, Soman, VX, Tabun or natural nervous toxins existing in animals or plants.

In the present invention, the term “a molecule with a specific ability to combine with an acetylcholinesterase inhibitor” refers to a molecule with an ability to identify acetylcholinesterase inhibitors, and the molecule has a specific structure to bind with acetylcholinesterase inhibitors. The examples of the molecules are, but not limited to, acetylcholinesterase or a compound with a structure similar to the acetylcholinesterase.

In the present invention, the term “inhibit” acetylcholinesterase refers to reduce, decrease, eliminate, diminish, slow or stop the acetylcholine hydrolysis reaction of acetylcholinesterases.

The present invention utilizes the surface modification technology to provide a chip for acetylcholinease inhibitor detection, said chip comprises: a substrate; and a film including a molecule with a specific ability to combine with an acetylcholinesterase inhibitor and said film is coated on said substrate. Specifically, the film of the above-mentioned chip was immobilized on the substrate through coupling reaction, wherein the coupling reaction could be carried out by any methods known in the field.

In one embodiment, the substrate of the chip for acetylcholinesterase inhibitor detection is a gold-coated substrate. The examples of substrate materials include, but not limited to, glass, quartz, silicon, plastic, metal, wafer or polymers. The molecule with a specific ability to combine with an acetylcholinesterase inhibitor is preferably acetylcholinesterase or molecules with a structure similar to the acetylcholinesterase.

The chip for acetylcholinesterase inhibitor detection of the invention can be prepared by any method known by a person skilled in the art. In a preferred embodiment, the chip is prepared by self-assembly monolayer technology: first, deposit a gold film on a substrate; form a sulfide self-assembly monolayer on the gold film; immobilize a molecule with a specific ability to combine with an acetylcholinesterase inhibitor, such as acetylcholinesterase, on the substrate by chemical reactions, and a chip for acetylcholinesterase inhibitor detection is then acquired.

In the other preferred embodiment, the substrate of the chip for acetylcholinesterase inhibitor detection can further be modified with dextran to obtain a hydrophilic surface. Molecules with a specific ability to combine with acetylcholinesterase inhibitors, such as acetylcholinesterase, are subsequently immobilized on the substrate by chemical reactions to acquire a chip for acetylcholinesterase inhibitor detection.

The chip of the invention utilizes the molecules with a specific binding ability for acetylcholinesterase inhibitors to identify acetylcholinesterase inhibitors. When the chip is exposed to an acetylcholinesterase inhibitor-contained environment, the molecule with a specific binding ability will identify the acetylcholinesterase inhibition molecules and combine with them, so that the acetylcholinesterase inhibition molecules will be immobilized on the chip. Through proper instruments, the adsorption of acetylcholinesterase inhibitors can be recognized and the concentrations of the inhibitors can be quantified. Accordingly, the present invention further provides an apparatus for acetylcholinesterase inhibitor detection, comprising: an acetylcholinesterase inhibitor detection chip including a substrate and a film coated on said substrate, wherein said film including a molecule with a specific ability to combine with a acetylcholinesterase inhibitor; and a detection element for detecting the surface variation of said acetylcholinease inhibitor detection chip.

In a preferred embodiment of the apparatus of the invention, the detection element is surface plasmon resonance. In this embodiment, the substrate of the acetycholinesterase inhibitor detection chip is a gold-coated substrate that is light transmissible, for example, but not limited to, gold-coated glass substrate or gold-coated quartz substrate. The detection principal of the surface plasmon resonance is described below. An incident light is first provided to the surface of gold-coated substrate, which is light transmissible, for inducing surface plasmon resonance. When there are molecules bound to the gold film surface, the phenomenon of surface plasmon resonance will be influenced, the dielectric of gold films changes and the refractive angle and the intensity of refracted light are influenced. Therefore, the adsorption of molecules on the gold film surface and their weights can be determined by detecting the variations of refractive angles and intensity of the refracted light.

In a preferred embodiment, the detection element is quartz crystal microbalance. In this embodiment, the substrate of the acetycholinesterase inhibitor detection chip is a gold-coated quartz substrate. Quartz crystal microbalance detects mass variation through measuring the change of oscillation frequency. When alternating current pass through a piezoelectric quartz crystal, a crystal deformation occurs and then crystal vibration is induced. Therefore, when the combination of an acetylcholinesterase inhibitor and a molecule immobilized on the detection chip occurs due to their structures or chemical reactions, the mass of the detection chip increases, resulting in the decrease of crystal vibration, whereby the mass of the sample can be calculated. The mass calculation equation is shown as equation (1):

$$Δ {m}_{QCM} = {ρ}_{f} {δ}_{f} = \frac{{C}_{QCM}}{n} Δ f \qquad ( 1 )$$

wherein Δm_{QCM} is the mass differential of the QCM chip, ρ_{f} is the increase of film density, δ_{f} is the increase of film thickness, n is a constant, C_{QCM} is the mass sensitivity of the QCM, and Δf is the frequency differential of crystal vibration. Under an ideal condition, the accuracy of the QCM can reach to ˜10⁻⁹ g/cm², so that the acetylcholinesterase inhibitor detection apparatus using QCM as a detection element provides extremely high sensitivity.

The acetylcholinesterase inhibitor detection chip and apparatus of the invention can detect the presence of acetylcholinesterase inhibitors. The detection steps are described below. First, provide an acetylcholinesterase inhibitor detection chip including a substrate and a film coated on said substrate, wherein said film including molecules with a specific ability to combine with acetylcholinesterase inhibitors. Subsequently, expose said acetylcholinesterase inhibitor detection chip to a sample, wherein the sample can be gas or liquid; when there are an acetylcholinesterase inhibitors contained in the sample, those acetylcholinesterase inhibitors would bind with the specific molecules immobilized on the film of the detection chip. After that, detect the surface variation of said acetylcholinesterase inhibitor detection chip with an appropriate instrument, such as SPR or QCM. Finally, analyze the detection results of the instrument to determine whether acetylcholinesterase inhibitors are contained in the sample and calculate their concentrations. Because the specific molecules included in the film of the detection chip has a specific ability to identify acetylcholinesterase inhibitors, there are only acetycholinesterase inhibitors captured by the specific molecules. Consequently, the detection results that show the adsorption of molecules on the chip surface refers to that there are acetylcholinesterase inhibitors contained in the sample.

The following examples are only used to illustrate the present invention, not intended to limit the scope thereof. Many modifications of the examples can be made without departing from the spirit of the present invention, and shall be included in the protection scope of claims of the invention.

### EXAMPLE 1

### Preparation of the Acetylcholinesterase Inhibitor Detection Chip

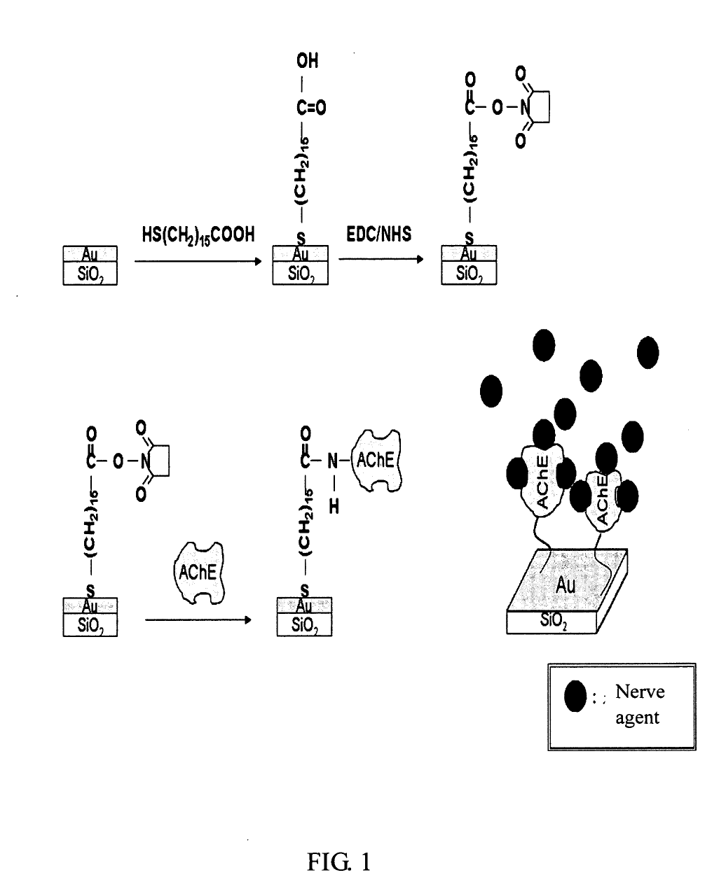

In this example, the substrate of the acetylcholinesterase inhibitor detection chip is glass. The process for preparation the chip is shown in FIG. 1. At first, the glass substrate was sputtered with a gold film and rinsed by 95% ethanol and deionized water (D.I. water) for several times. The gold-coated glass substrate was then dried with N₂ and placed in a solution of 1 mM HS(CH₂)₁₅COOH dissolved in 99.8% ethanol. After 2 hours, took the substrate out, rinsed it with 95% ethanol and D.I. water for 3 times at least, and dried with N₂. Then the substrate was placed in 1 mg/ml EDC solution with a volume of 1 ml (dissolved in 40 mM NHS solution) for 4 hours. After rinsed with D.I. water for 3 times, the substrate was subsequently reacted with 1 mM acetylcholinesterase in D.I. water for 24 hours, and then an acetylcholinesterase inhibitor detection chip was prepared.

### EXAMPLE 2

### Preparation of the Acetylcholinesterase Inhibitor Detection Chip

The example provides the other process for the preparation of the acetylcholinesterase inhibitor detection chip.

Solution Preparation:

Phosphate Buffer Saline (PBS): 28 ml of 0.2M NaH₂PO₄.H₂O solution was mixed with 72 ml of 0.2M Na₂HPO₄.H₂O, and D.I. water was added until the total volume was 200 ml. The pH value was adjusted with H₃PO₄/NaOH. A PBS solution with 0.1M and pH 7.0 was then prepared.

Ethylenediamine solution: 33 μl of 15M ethylenediamne was dissolved in 10 ml D.I. water and the pH value was adjusted with HNO₃ to pH 8.0. An ethylenediamine solution with 50 mM and pH 8.0 was then prepared.

Ethanolamine solution: 61.08 μl of ethanolamine was dissolved in 10 ml D.I. water and the pH value was adjusted with HCl to pH 8.0. An ethanolamine solution with 0.1M and pH 8.0 was then prepared.

HS(CH₂)₁₅COOH solution: 1 mM HS(CH₂)₁₅COOH solution was prepared in 99.8% ethanol.

EDC solution: 0.767 g EDC was dissolved in 10 ml D.I. water to obtain 0.4 M EDC solution.

NHS solution: 0.46 g NHS was dissolved in 10 ml D.I. water to obtain 0.4M NHS solutions.

POD solution: 100 mg dextran was dissolved in 50 ml D.I. water and 398 mg NalO₄ was added. After stirred for 2 hours, the mixtures was loaded in dialysis membrane with MWCO 12,000˜14,000 and dialyzed with 5 L D.I. water for 3 times.

Nitrilotriacetic acid (NTA) solution: 0.0146 g NTA was dissolved in 5 ml PBS to obtain 0.011M NTA solutions.

POD/NTA solution: 0.0146 g NTA was dissolved in 1 ml POD solution, wherein the molar ratio of POD: NTA was 1:5.

NaCNBH₃ solution: 0.31 g NaCNBH₃ was dissolved in 5 ml of 1N NaOH solution to obtain 1 M NaCNBH₃ solution.

Chip Preparation:

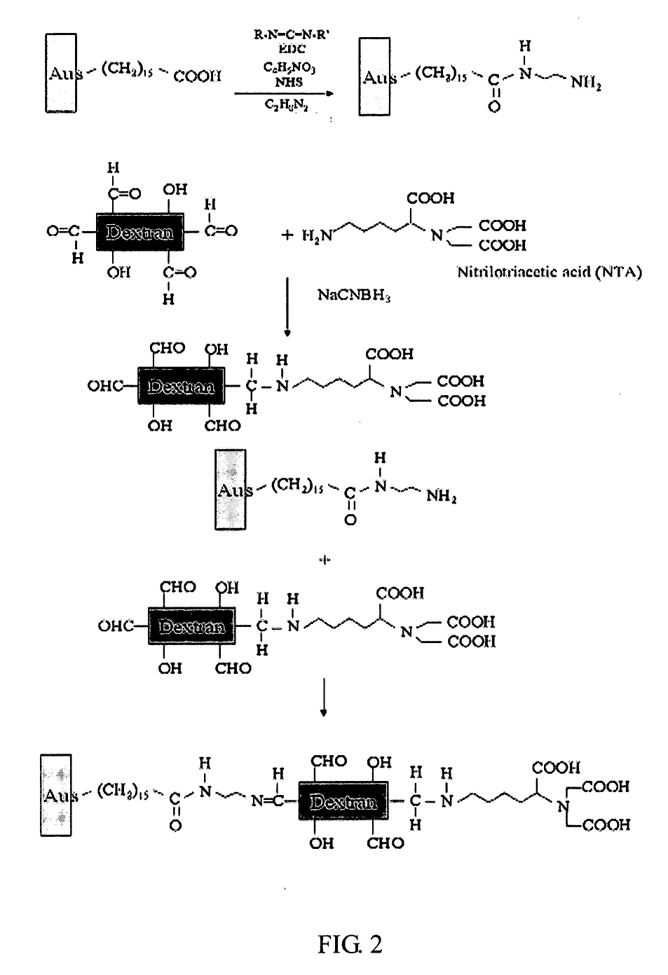

As shown in FIG. 2, a glass substrate was sputtered with a gold film at first and rinsed with 95% ethanol and D.I, water for several times. The substrate was then immersed in a solution of 1 mM HS(CH₂)₁₅COOH dissolved in 99.8% ethanol for 2 hours. Subsequently, the substrate was taken out, rinsed with ethanol for several times, dried with N₂, and immersed in a solution of EDC/NHS mixed with equal volume. After 2 hours, took the substrate out, rinsed with D.I. water for several times and dried with N₂. The substrate was then immersed in 0.1 M ethanolamine solution for 1 hour, POD/NTA solution for 2 hours, rinsed with D.I. water for several times and dried with N₂. A NaCNBH₃ reductant was added into POD/NTA solution before the substrate was immersed. Following the procedure above, a hydrophilic surface with dextran on the chip surface was then acquired.

The substrate modified with dextran was rinsed with 95% ethanol and D.I. water for three times, dried with N₂, then placed in 1 mg/ml EDC solution with a volume of 1 ml (dissolved in 40 mM NHS solution) for 4 hours. After rinsed with D.I. water for 3 times, the substrate was then reacted with 1 mM acetylcholinesterase in D.I. water for 24 hours. Consequently, an acetylcholinesterase inhibitor detection chip was prepared.

### EXAMPLE 3

### Detection and Quantification of Acetyicholinesterase Inhibitor by SPR

The example is one embodiment of the acetylcholinesterase inhibitor detection apparatus of the invention. In this example, SPR is used as a detection element to detect the surface variation of the acetylcholinesterase inhibitor detection chip.

First, the chip prepared according to the process of the example 1 or example 2 was exposed to a sample for a period, and then put on the triangle prism of the SPR. After opening a light source, an incident light passed through a polarization apparatus to be polarized. The polarized light then passed through the triangle prism and reached the chip surface. A surface plasmon resonance in the gold film of the chip was then generated, leading to the changes of the refractive angle and the intensity of the refracted light. The refracted light was refracted by a triangle prism and then detected by a detector to record the refractive angle and light intensity. By analyzing the records of light intensity and refractive angles, the adsorption of molecules on the detection chip could be determined and the mass of those molecules adsorbed on the gold film could be calculated.

SPR can detect variations of the dielectric constant caused by interactions of biomolecules in the interface of solid and liquid or the interface of solid and gas. In this example, the molecule immobilized on the detection chip surface is acetylcholinesterase, so that only the molecule with a structure capable of binding with an acetylcholinesterase would be adsorbed on the detection chip surface. In other words, only the acetylcholinesterase inhibitor would be adsorbed on the detection chip. When a molecule adsorbed on the gold film of the detection chip, the dielectric constant of the gold film changes, influencing the refractive angles and the intensity of the refracted light. Therefore, the presence and the concentrations of the acetylcholinesterase inhibitor can be determined according to the SPR records of refractive angles and the intensity of refracted light.

In this example, in addition to the measurement of light intensity and refractive angle, the presence of adsorbed molecules can also be determined by wavelength detection or phase shift measurement depending on interference.

### EXAMPLE 4

### Preparation of the Acetyicholinesterase Inhibitor Detection Chip

In this example, the substrate of the acetylcholinesterase inhibitor detection chip is piezoelectric quartz crystal coated with gold electrodes. The preparation process is the same as example 1. At first, the piezoelectric quartz crystal was rinsed with 95% ethanol and D.I. water for several times and dried with N₂, then immersed in a solution of 1 mM HS(CH₂)₁₅COOH dissolved in 99.8% ethanol. After 2 hours, the piezoelectric quartz crystal was rinsed with 95% ethanol and D.I. water for 3 times at least, dried with N₂ and then immersed in a 1 mg/ml EDC solution with 1 ml (dissolved in 40 mM NHS solution) for 4 hours. After rinsed with D.I. water for 3 times, the quartz crystal was reacted with 1 mM acetylcholinesterase in D.I. water for 24 hours. The piezoelectric quartz crystal chip for acetylcholinesterase inhibitor detection was then prepared.

### EXAMPLE 5

### Preparation of the Acetylcholinesterase Inhibitor Detection Chip

In this example, the substrate of the acetylcholinesterase inhibitor detection chip is a piezoelectric quartz crystal coated with gold electrodes. The preparation process is the same as example 2

At the beginning, the quartz crystal was rinsed with 95% ethanol and D.I. water for several times, dried with N₂ and then placed in the solution of 1 mM HS(CH₂)₁₅COOH dissolved in 99.8% ethanol for 2 hours. After that, the piezoelectric quartz crystal was rinsed with ethanol again, dried with N₂, and then immersed in a solution of EDC/NHS mixed with equal volume for 2 hours. After rinsed with D.I. water for several times, dried with N₂, the quartz crystal was immersed in 0.1 M ethanolamine solution for 1 hour and POD/NTA solution for 2 hours, wherein the POD/NTA solution was added with NaCNBH₃ reductant before the quartz crystal immersed. The modified quartz crystal was then taken out, rinsed with D.I. water for several times and dried with N₂. Following the procedure above, a hydrophilic surface with dextran on the chip surface was then acquired.

The quartz crystal modified with dextran was rinsed with 95% ethanol and D.I. water for three times, dried with N₂, then placed in 1 ml EDC solution with the concentration of 1 mg/ml (dissolved in 40 mM NHS solution) for 4 hours. After rinsed by D.I. water for 3 times, the quartz crystal was then reacted with 1 mM acetylcholinesterase in D.I. water for 24 hours. Accordingly, the piezoelectric quartz crystal chip for acetylcholinesterase inhibitor detection was then prepared.

### EXAMPLE 6

### Detection and Quantification of Acetylcholinesterase Inhibitor by QCM

This example is the other embodiment of the acetylcholinesterase inhibitor detection apparatus of the invention. In this example, a QCM is used as a detection element of the acetylcholinesterase inhibitor detection apparatus.

The piezoelectric quartz crystal detection chip prepared according to the process in example 4 or example 5 was put in a testing chamber of the QCM, wherein the gold electrode of the piezoelectric quartz crystal was connected with an electron oscillator circuit and a frequency calculator. A sample was then injected into the testing chamber to contact with the detection chip. When there were acetycholinesterase inhibition molecules contained in the sample, the molecules would combined with the acetylcholinesterases immobilized on the gold electrode of the piezoelectric quartz crystal, increasing weights on the electrode surface, changing the oscillation frequency of the piezoelectric quartz crystal. Therefore, the presence and concentrations of acetylcholinesterase inhibition molecules can be determined from the oscillation frequency variation of the piezoelectric quartz crystal.

Because the oscillation frequency of a piezoelectric quartz crystal depends on the weight loading on the crystal, the QCM measures the weight of the sample by recording the oscillation frequency variation. Piezoelectric quartz crystal is a signal transduction element of a piezoelectric sensor. It consists of a quartz crystal sandwiched between a pair of gold electrodes and converts detected results into electronic signals for transmission and amplification. The gold electrode is primarily used to conduct an oscillating electric field perpendicular to the crystal surface into the crystal and a mechanical oscillation in the crystal interior then occurs due to the inverse piezoelectric effect. If the thickness of a quartz crystal is constant, the mechanical oscillation will be generated at a constant resonance frequency. The resonance frequency can be measured by utilizing a proper electron oscillator circuit. According to the Sauerbrey equation, the mass variation of the electrode surface (Δm) has a linear relationship to the oscillation frequency variation of the crystal (ΔF), that is, ΔF∝Δm, for an AT-cut quartz crystal.

The present example modified the gold electrode of the piezoelectric quartz crystal with a specific molecule capable of identifying acetylcholinesterase inhibitor and exposed the modified crystal to a sample. When an acetylcholinesterase inhibitor contained in the sample, an acetylcholinesterase inhibition molecule would adsorb on the gold electrode by combining with the acetylcholinesterase on the gold electrode. Through the measurement of the variation of oscillation frequency, the adsorption of acetylcholinesterase inhibitor molecules and its weight can be determined.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A chip for acetylcholinesterase inhibitor detection, comprising:
a substrate; and
a film including a molecule with a specific ability to combine with an acetylcholinesterase inhibitor and said film is coated on said substrate.

2. The chip for acetylcholinesterase inhibitor of claim 1, wherein said substrate is coated with gold.

3. The chip for acetylcholinesterase inhibitor of claim 1, wherein the material of said substrate comprise glass, quartz, silicon, plastic, metal or polymer.

4. The chip for acetylcholinesterase inhibitor of claim 1, wherein said molecule with a specific ability to combine with an acetylcholinesterase inhibitor is acetylcholinesterase or a compound with a structure similar to an acetylcholinesterase.

5. The chip for acetylcholinesterase inhibitor of claim 1, wherein said acetylcholinesterase inhibitor is any natural or synthesized compound or molecule that influence the acetylcholine hydrolysis function of acetylcholinesterase.

6. The chip for acetylcholinesterase inhibitor of claim 5, wherein said acetylcholinesterase inhibitor comprise nerve agents, organophosphorus pesticides or medicines for aceylcholinesterase inhibition.

7. The chip for acetylcholinesterase inhibitor of claim 1, wherein said chip is used to detect gas or liquid acetylcholinesterase inhibitors.

8. An apparatus for acetylcholinesterase inhibitor detection, comprising:
an acetylcholinesterase inhibitor detection chip including a substrate and a film coated on said substrate, wherein said film including a molecule with a specific ability to combine with an acetylcholinesterase inhibitor; and
a detection element for detecting the surface variation of said acetylcholinesterase inhibitor detection chip.

9. The apparatus for acetylcholinesterase inhibitor detection of claim 8, wherein said molecule with a specific ability to combine with a acetylcholinesterase inhibitor is acetylcholinesterase or a compound with a structure similar to an acetylcholinesterase.

10. The apparatus for acetylcholinesterase inhibitor detection of claim 8, wherein said detection element is surface plasmon resonance.

11. The apparatus for acetylcholinesterase inhibitor detection of claim 10, wherein said substrate of the chip is light transmissible and which is coated with gold.

12. The apparatus for acetylcholinesterase inhibitor detection of claim 8, wherein said detection element is quartz crystal microbalance.

13. The apparatus for acetylcholinesterase inhibitor detection of claim 12, wherein said substrate is a piezoelectric quartz crystal coated with gold electrodes.

14. The apparatus for acetylcholinesterase inhibitor detection of claim 8, wherein said apparatus is used to detect gas or liquid acetylcholinesterase inhibitors.

15. The apparatus for acetylcholinesterase inhibitor detection of claim 8, wherein said apparatus is capable of quantification of molecules adsorbed on said acetylcholinesterase inhibitor detection chip.

16. A method for detecting acetylcholinesterase inhibitors, comprising the following steps of:
(a) providing an acetylcholinesterase inhibitor detection chip including a substrate and a film coated on said substrate, wherein said film including a molecule with a specific ability to combine with an acetylcholinesterase inhibitor;
(b) contacting said acetylcholinesterase inhibitor detection chip with a sample;
(c) detecting the surface variation of said acetylcholinesterase inhibitor detection chip; and
(d) determining whether an acetylcholinesterase inhibitor exists in the sample.

17. The method for detecting acetylcholinesterase inhibitors of claim 16, wherein said sample of step (b) is gas sample or liquid sample.

18. The method for detecting acetylcholinesterase inhibitors of claim 16, wherein said step (c) is to detect the surface variation of said acetylcholinesterase inhibitor detection chip by surface plasmon resonance or quartz crystal microbalance.

19. The method for detecting acetylcholinesterase inhibitors of claim 16, which further comprises a step of quantifying the molecules adsorbed on said acetylcholinesterase inhibitor detection chip.
